(19) United States
(12) Patent Application Publication
(10) Pub. No.: US 2017/0119254 A1
(43) Pub. Date: May 4, 2017

(54) CIRCULATION TIME MEASUREMENT DEVICE, ESTIMATED CARDIAC OUTPUT CALCULATION APPARATUS, CIRCULATION TIME MEASUREMENT METHOD, ESTIMATED CARDIAC OUTPUT CALCULATION METHOD, AND PROGRAM

(71) Applicant: KYUSHU UNIVERSITY, NATIONAL UNIVERSITY CORPORATION, Fukuoka-shi, Fukuoka (JP)

(72) Inventors: Shinichi ANDO, Kasuga-shi (JP); Kazuya HOSOKAWA, Fukuoka-shi (JP)

(73) Assignee: KYUSHU UNIVERSITY, NATIONAL UNIVERSITY CORPORATION, Fukuoka-shi, Fukuoka (JP)

(21) Appl. No.: 15/317,604

(22) PCT Filed: Jun. 5, 2015
(86) PCT No.: PCT/JP2015/066341

§ 371 Date: Dec. 9, 2016

Related U.S. Application Data
(60) Provisional application No. 62/011,590, filed on Jun. 13, 2014.

(51) Int. Cl. A61B 5/0205; A61B 5/00; A61B 5/026

(52) U.S. Cl. CPC A61B 5/0205; A61B 5/026; A61B 5/7246; A61B 5/7278; A61B 5/087

(57) ABSTRACT

A circulation time measurement device includes: a signal acquisition unit that acquires an air flow signal indicating a temporal change of an air flow of breathing and an oxygen saturation signal indicating a temporal change of oxygen saturation; and a circulation time calculation unit that measures an oxygen delivery circulation time of blood based on a time difference between a predetermined first time in the air flow signal and a second time in the oxygen saturation signal indicating an increase in oxygen saturation corresponding to resumption of breathing at the first time.

Priority is claimed on U.S. Provisional Patent Application No. 62/011,590, filed on Jun. 13, 2014, the content of which is incorporated herein by reference.

### TECHNICAL FIELD

The present invention relates to a circulation time measurement device, an estimated cardiac output calculation apparatus, a circulation time measurement method, an estimated cardiac output calculation method, and a program.

### BACKGROUND ART

There is a cardiac output as one of indicators indicating the state of a heart function. The cardiac output indicates the amount of blood ejected from the heart in one minute, and this value is reduced if there is a decline in heart function. There are various methods of measuring the cardiac output. As a typical measurement method, for example, there is a thermal dilution method. In addition to this, MRI, echocardiography, an impedance method, and the like are provided.

A person whose heart function has declined is said to have a problem in breathing in many cases. For example, it has been pointed out that a patient with heart failure has sleep apnea at a high rate. In addition, studies show that, in a case where the breathing of a subject with sleep apnea is resumed from the state of apnea, a time from the resumption of the breathing to an increase in oxygen saturation (SpO₂) in blood is correlated with an indicator of the heart function (NPL 1).

As a related technique, PTL 1 discloses a measurement method capable of measuring the circulation time of the oxygen delivery of the blood flow in a non-invasive manner. In addition, PTL 1 discloses that the circulation time of the oxygen delivery of the blood flow is well correlated with the cardiac output.

### CITATION LIST

### Patent Literature

[PTL 1] Japanese Unexamined Patent Application, First Publication No. 2006-231012

### Non-Patent Literature

[NPL 1] M. J. Hall, A. Xie, R. Rutherford, et al., “Cycle length of periodic breathing in patients with and without heart failure” Am. J. Respir. Crit. Care Med., 154, 376-381, 1996.

### SUMMARY OF INVENTION

### Technical Problem

Incidentally, the thermal dilution method that is currently used in many cases is an invasive method of inserting a catheter into the heart. Accordingly, various problems, such as a physical burden on a subject, have been pointed out. In addition, in a measurement method, such as echocardiography, there is a problem that the accuracy cannot be maintained. In addition, although it has been pointed out that there is a strong correlation between sleep apnea and heart function, a simple and practical method of measuring the cardiac output using the correlation has not yet been provided. There is no description regarding such a method in PTL 1.

Therefore, it is an object of the present invention to provide a circulation time measurement device, an estimated cardiac output calculation apparatus, a circulation time measurement method, an estimated cardiac output calculation method, and a program capable of solving the aforementioned problem.

### Solution to Problem

According to a first aspect of the present invention, a circulation time measurement device includes: a signal acquisition unit that acquires an air flow signal indicating a temporal change of an air flow of breathing and an oxygen saturation signal indicating a temporal change of oxygen saturation; and a circulation time calculation unit that measures an oxygen delivery circulation time of blood based on a time difference between a predetermined first time in the air flow signal and a second time in the oxygen saturation signal indicating an increase in oxygen saturation corresponding to resumption of breathing at the first time.

According to a second aspect of the present invention, the circulation time calculation unit shapes the air flow signal into a waveform showing a period of stop and resumption of breathing, and measures the oxygen delivery circulation time based on a time lag between the waveform after shaping and a waveform indicated by the oxygen saturation signal.

According to a third aspect of the present invention, the circulation time calculation unit includes: an air flow segment section that segments the air flow signal at predetermined time intervals to generate a segmental air flow signal; an oxygen saturation segment section that segments the oxygen saturation signal at predetermined time intervals to generate a segmental oxygen saturation signal; a signal shaping processing section that generates a shaped segmental air flow signal by shaping the segmental air flow signal into a waveform showing a period of stop and resumption of breathing by applying a filter to the segmental air flow signal; and a time difference calculation section that calculates a time difference corresponding to a time lag between a waveform indicated by the shaped segmental air flow signal and a waveform indicated by the segmental oxygen saturation signal and sets the time difference as the oxygen delivery circulation time.

According to a fourth aspect of the present invention, the time difference calculation section calculates the time difference using a cross-correlation analysis for the shaped segmental air flow signal and the segmental oxygen saturation signal.

According to a fifth aspect of the present invention, an estimated cardiac output calculation apparatus includes: the circulation time measurement device described in any one of the first to fourth aspects; and a cardiac output calculation unit that acquires the oxygen delivery circulation time measured by the circulation time measurement device and calculates an estimated cardiac output based on the acquired oxygen delivery circulation time and a predetermined hyperbolic function showing a relationship between the oxygen delivery circulation time of blood and a cardiac output.

According to a sixth aspect of the present invention, a circulation time measurement method includes: a step of acquiring an air flow signal indicating a temporal change of an air flow of breathing and an oxygen saturation signal indicating a temporal change of oxygen saturation; and a step of measuring an oxygen delivery circulation time of blood based on a time difference between a predetermined first time in the air flow signal and a second time in the oxygen saturation signal indicating an increase in oxygen saturation corresponding to resumption of breathing at the first time.

According to a seventh aspect of the present invention, an estimated cardiac output calculation method includes a step of acquiring an air flow signal indicating a temporal change of an air flow of breathing and an oxygen saturation signal indicating a temporal change of oxygen saturation; a step of measuring an oxygen delivery circulation time of blood based on a time difference between a predetermined first time in the air flow signal and a second time in the oxygen saturation signal indicating an increase in oxygen saturation corresponding to resumption of breathing at the first time; and a step of calculating an estimated cardiac output based on the measured oxygen delivery circulation time and a predetermined hyperbolic function showing a relationship between the oxygen delivery circulation time of blood and a cardiac output.

According to an eighth aspect of the present invention, a program causes a computer of a circulation time measurement device to function as: means for acquiring an air flow signal indicating a temporal change of an air flow of breathing and an oxygen saturation signal indicating a temporal change of oxygen saturation; and means for measuring an oxygen delivery circulation time of blood based on a time difference between a predetermined first time in the air flow signal and a second time in the oxygen saturation signal indicating an increase in oxygen saturation corresponding to resumption of breathing at the first time.

According to a ninth aspect of the present invention, a program causing a computer of an estimated cardiac output calculation apparatus to function as: means for acquiring an air flow signal indicating a temporal change of an air flow of breathing and an oxygen saturation signal indicating a temporal change of oxygen saturation; means for measuring an oxygen delivery circulation time of blood based on a time difference between a predetermined first time in the air flow signal and a second time in the oxygen saturation signal indicating an increase in oxygen saturation corresponding to resumption of breathing at the first time; and means for calculating an estimated cardiac output based on the measured oxygen delivery circulation time and a predetermined hyperbolic function showing a relationship between the oxygen delivery circulation time of blood and a cardiac output.

### Advantageous Effects of Invention

According to the aspects of the present invention described above, it is possible to measure the oxygen delivery circulation time of blood and estimate the cardiac output using the breathing period of the subject and the time-series data of oxygen saturation in blood.

### BRIEF DESCRIPTION OF DRAWINGS

FIG. 1 is a block diagram showing the configuration of an estimated cardiac output calculation apparatus in one embodiment of the present invention.

FIG. 2 is a block diagram showing the configuration of a circulation time measurement device in one embodiment of the present invention.

FIG. 3 is a first diagram illustrating the outline of the circulation time measurement process in one embodiment of the present invention.

FIG. 4 is a second diagram illustrating the outline of the circulation time measurement process in one embodiment of the present invention.

FIG. 5 is a third diagram illustrating the outline of the circulation time measurement process in one embodiment of the present invention.

FIG. 6 is a diagram illustrating the calculation of a cardiac output in one embodiment of the present invention.

FIG. 7 is a flowchart of the process of calculating the cardiac output according to one embodiment of the present invention.

FIG. 8 is a first diagram showing an example of the graph output from the estimated cardiac output calculation apparatus in one embodiment of the present invention.

FIG. 9 is a second diagram showing an example of the graph output from the estimated cardiac output calculation apparatus in one embodiment of the present invention.

### DESCRIPTION OF EMBODIMENTS

### One Embodiment

Hereinafter, an estimated cardiac output calculation apparatus according to an embodiment of the present invention will be described with reference to the diagrams.

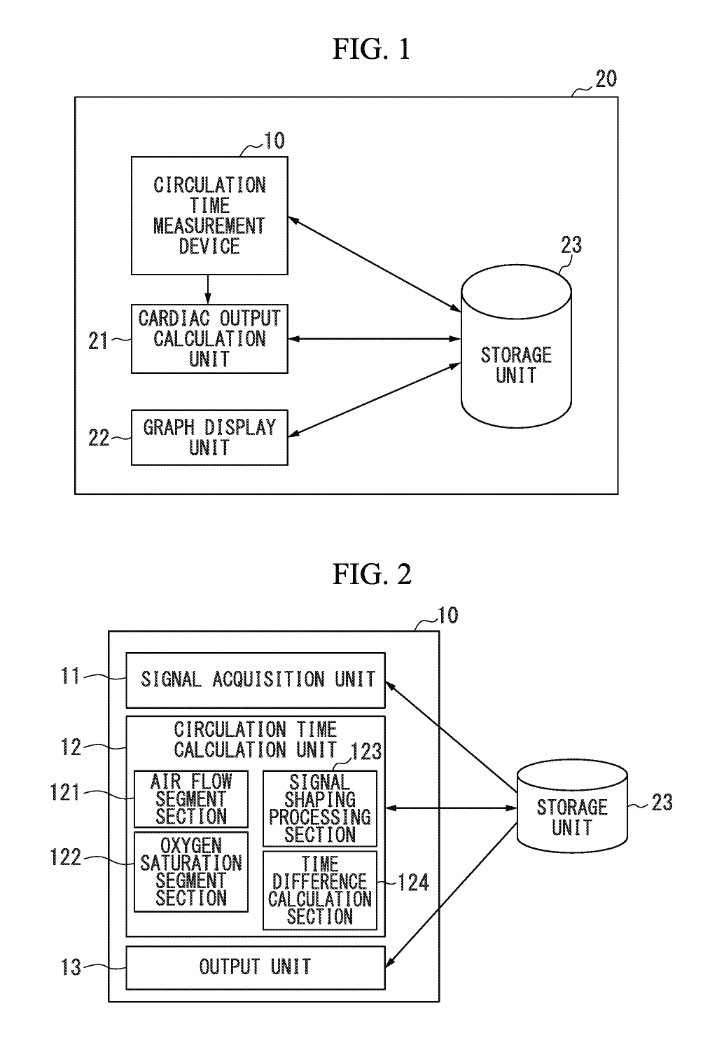

FIG. 1 is a block diagram showing the configuration of the estimated cardiac output calculation apparatus in one embodiment of the present invention.

The estimated cardiac output calculation apparatus 20 is an apparatus for calculating the estimated value of the cardiac output of a subject. The cardiac output is, for example, a cardiac index (CI). Alternatively, it is also possible to use CO (cardiac output: CO/body surface area=CI). In the following explanation, a case where the CI is used as the cardiac output will be described as an example. The estimated cardiac output calculation apparatus 20 of the present embodiment is an apparatus capable of calculating the estimated value of the cardiac output with high accuracy without requiring expensive and special medical equipment. The estimated cardiac output calculation apparatus 20 is a personal computer (PC) or a server device including a central processing unit (CPU), for example. The estimated cardiac output calculation apparatus 20 is connected to a display device, a keyboard, a mouse, and the like.

In FIG. 1, the estimated cardiac output calculation apparatus 20 includes a circulation time measurement device 10, a cardiac output calculation unit 21, a graph display unit 22, and a storage unit 23.

The circulation time measurement device 10 measures the oxygen delivery circulation time of the blood of the subject using an air flow signal, which is measured by an air flow sensor for detecting the state of breathing, and an oxygen saturation signal, which is measured by a sensor for detecting oxygen saturation in blood. The oxygen delivery circulation time of blood is a time from the start of breathing of a subject to a time, at which blood oxygenated by oxygen inhaled by the breathing is delivered by the blood flow to reach a predetermined position.

The cardiac output calculation unit 21 acquires the circulation time measured by the circulation time measurement device 10, and calculates a cardiac output using the circulation time. In the case of normal subjects, there is no problem in the amount of blood supplied from the heart. In this case, the oxygen delivery circulation time of blood indicates a normal value. However, in the case of patients who have a problem in a heart function, the action of the heart is weak, and the amount of blood supplied from the heart is smaller than that in normal subjects. Accordingly, since a time is required until oxygen reaches a predetermined position, the oxygen delivery circulation time of blood is longer than values in normal subjects. Although the correlation between the oxygen delivery circulation time of blood and the cardiac output is known up to now, there is no information regarding the exact relationship between the oxygen delivery circulation time of blood and the cardiac output. In the present embodiment, a method of calculating the accurate estimated value of the cardiac output from the oxygen delivery circulation time of blood using an expression showing the correlation between the oxygen delivery circulation time of blood and the cardiac output is provided.

The graph display unit 22 creates a graph of the time-series of the estimated value of the cardiac output calculated by the cardiac output calculation unit 21, and outputs the graph to a display device or the like connected to the estimated cardiac output calculation apparatus 20.

The storage unit 23 stores various kinds of information, such as a function required for the calculation of the cardiac output, an air flow signal, and an oxygen saturation signal.

FIG. 2 is a block diagram showing the configuration of the circulation time measurement device in one embodiment of the present invention.

The circulation time measurement device 10 is a device for measuring the oxygen delivery circulation time of blood of the subject. The circulation time measurement device 10 of the present embodiment measures the oxygen delivery circulation time of blood based on the state of breathing during sleep of a subject suffering from sleep-disordered breathing and a change in oxygen saturation in the blood at the time. In the case of a subject suffering from sleep-disordered breathing, there is a time, at which breathing is stopped or weakened, during sleep. In the meantime, the oxygen saturation of blood of the subject is reduced. Then, if the subject resumes breathing, the oxygen saturation of blood rises. In the case of a subject suffering from sleep-disordered breathing, resumption of breathing or an increase in oxygen saturation of the blood due to the resumption of breathing clearly appears in the air flow signal or the oxygen saturation signal measured for the subject. The circulation time measurement device 10 of the present embodiment measures the oxygen delivery circulation time of blood using such a characteristic.

In the present embodiment, it is possible to use the air flow signal and the oxygen saturation signal of the subject that have been measured by polysomnography. The air flow signal can be measured by using a pressure sensor attached to the nose of the subject, for example. Besides, in order to measure the air flow signal, it is possible to use a method of detecting the temperature change of the air that goes in and out of the nasal cavity with breathing, a method of detecting the movement of the chest due to breathing, and the like. In addition, the oxygen saturation signal can be measured using a pulse oximeter attached to the fingertip of the subject for example. In the present embodiment, as the oxygen delivery circulation time of blood, a lung-to-finger circulation time (LFCT) that means a time for which oxygen is delivered from the lung to the fingertip of the subject is used.

As shown in FIG. 2, the circulation time measurement device 10 includes a signal acquisition unit 11, a circulation time calculation unit 12, and an output unit 13.

The signal acquisition unit 11 acquires an air flow signal indicating the temporal change of the breathing state of the subject from the storage unit 23. In addition, the signal acquisition unit 11 acquires an oxygen saturation signal indicating the temporal change of the oxygen saturation in blood, which flows through the fingertip of the subject, from the storage unit 23.

The circulation time calculation unit 12 measures the oxygen delivery circulation time of blood based on the time difference between the first time (resumption time of breathing) in the air flow signal and the second time in the oxygen saturation signal, which indicates the behavior of oxygen saturation (increase in oxygen saturation) corresponding to the resumption of breathing at the first time. In this calculation, the circulation time calculation unit 12 shapes the air flow signal into a waveform showing the period of stop and resumption of breathing, and measures the oxygen delivery circulation time of blood based on a time lag between the waveform after shaping and the waveform indicated by the oxygen saturation signal.

The output unit 13 outputs the information of the oxygen delivery circulation time of blood calculated by the circulation time calculation unit 12.

The circulation time calculation unit 12 includes an air flow segment section 121, an oxygen saturation segment section 122, a signal shaping processing section 123, and a time difference calculation section 124.

The air flow segment section 121 segments the air flow signal at predetermined time intervals to generate a segmental air flow signal.

The oxygen saturation segment section 122 segments the oxygen saturation signal at predetermined time intervals to generate a segmental oxygen saturation signal.

The signal shaping processing section 123 generates a shaped segmental air flow signal, for example, by applying a low pass filter to the segmental air flow signal.

The time difference calculation section 124 calculates a time difference corresponding to the time lag between the waveform indicated by the shaped segmental air flow signal and the waveform indicated by the segmental oxygen saturation signal, and sets the time difference as the oxygen delivery circulation time of blood. In calculating the time difference, the time difference calculation section 124 uses a cross-correlation analysis for the shaped segmental air flow signal and the segmental oxygen saturation signal.

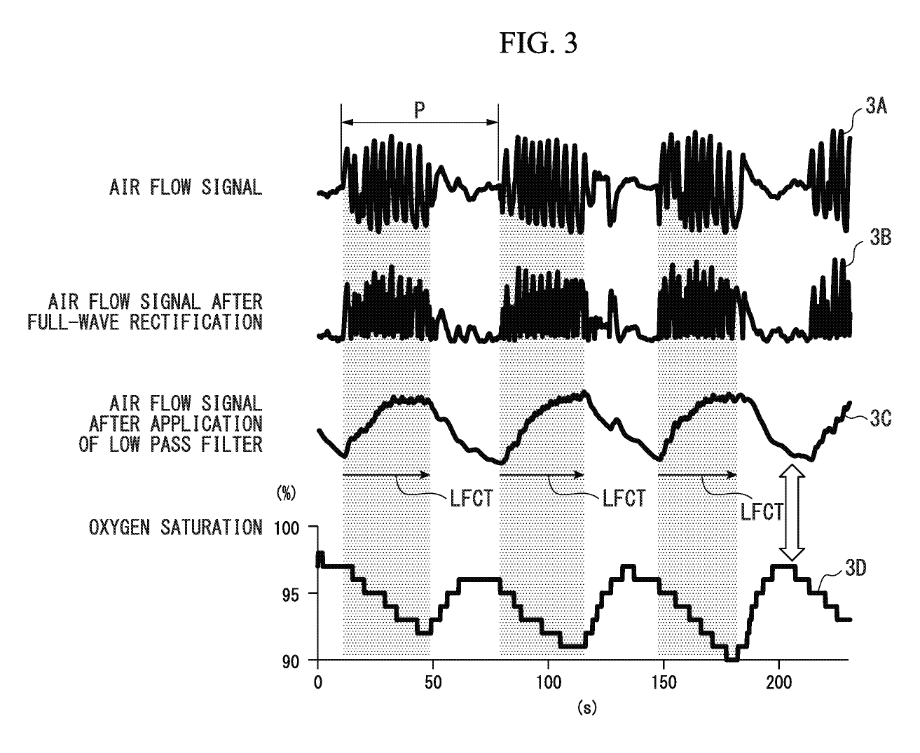

FIG. 3 is a first diagram illustrating the outline of the circulation time measurement process in one embodiment of the present invention.

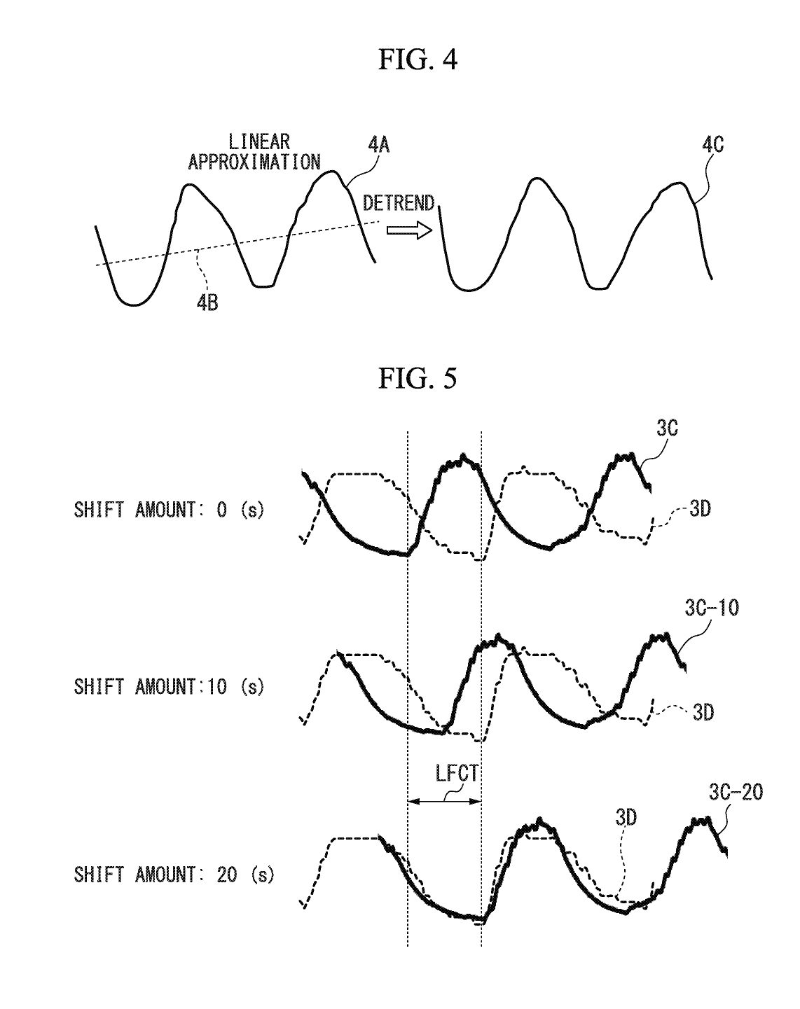

FIG. 4 is a second diagram illustrating the outline of the circulation time measurement process (detrend processing) in one embodiment of the present invention.

A graph 3A (uppermost graph) shown in FIG. 3 is a graph of the time-series of the air flow signal acquired by the signal acquisition unit 11. A graph 3B (second graph from the top) shown in FIG. 3 is a graph of the time-series of a signal obtained by performing full-wave rectification processing on the air flow signal acquired by the signal acquisition unit 11. A graph 3C (third graph from the top) shown in FIG. 3 is a graph of the time-series of a signal that is shaped into a waveform showing the period of stop and resumption of breathing by applying a low pass filter to the air flow signal after full-wave rectification processing. A graph 3D (lowermost graph) shown in FIG. 3 is a graph of the time-series of the oxygen saturation signal acquired by the signal acquisition unit 11.

A patient with heart failure or the like also suffers from sleep-disordered breathing in many cases. In the present embodiment, the LFCT is measured using the timing at which the breathing of the subject suffering from sleep-disordered breathing returns to normal breathing from the stop or weakened state during sleep. Specifically, when the breathing of the subject returns from the stop state to the normal breathing state, oxygen saturation in blood is increased by oxygen inhaled at that time. An increase in oxygen saturation is recorded with a slight delay in the oxygen saturation signal. The reason for the slight delay is that the time is required until oxygenated blood is delivered to the fingertip of the subject. Also in one air flow signal, when the breathing of the subject returns from the stop state to the normal breathing state, the behavior is clearly shown. In the present embodiment, the LFCT is measured using the behavior of the air flow signal at the time of resumption of breathing in a series of breathing and the behavior of oxygen saturation corresponding thereto as marks, with a subject whose breathing may be stopped during sleep as a target. The LFCT is a time until the pulse oximeter attached to the fingertip of the subject detects an increase in oxygen saturation from the start of inhaling of oxygen. In the present embodiment, the time difference between the time, at which a behavior indicating the resumption of breathing in the air flow signal appears, and the time, at which a behavior corresponding to the resumption of breathing appears in the oxygen saturation signal, is calculated by analyzing the cross-correlation between the waveforms indicated by the respective signals.

In the air flow signal indicated by the graph 3A, a breathing waveform having a shorter period (higher frequency) than a period (P) of stop and resumption of breathing is included. Even if cross-correlation analysis is performed in this state, the cross correlation analysis cannot be correctly performed due to the influence of high-frequency signals. Therefore, processing for extracting only a waveform mainly indicating the period of stop and resumption of breathing is performed so that cross-correlation analysis between the air flow signal and the waveform indicated by the oxygen saturation signal can be easily performed.

First, the signal shaping processing section 123 performs full-wave rectification processing. Therefore, as shown in the graph 3B, all values of the air flow signal become positive values. Then, the signal shaping processing section 123 removes high-frequency components by applying a low pass filter to the air flow signal after full-wave rectification processing. Then, it is possible to extract only the waveform showing the period of stop and resumption of breathing indicated by the graph 3C. In this stage, detrend processing is performed in addition to frequency cut-off using a low pass filter.

The detrend processing will be described with reference to FIG. 4. When the air flow signal is expressed in a graph, there is a tendency that the value of the signal increases gradually due to the accumulation of noise in the air flow sensor or the like. A graph 4A shows an example of the air flow signal in such a case. It is not possible to perform correct cross-correlation analysis under such an increase tendency. The detrend processing is performed in order to extract only the waveform indicating the period of stop and resumption of breathing by removing such a tendency of the value. The signal shaping processing section 123 calculates a straight line 4B showing the increase tendency of the air flow signal by performing linear approximation for the air flow signal using a least squares method, for example. The signal shaping processing section 123 performs detrend processing for subtracting a value corresponding to the straight line from the value of the air flow signal. A graph 4C shows a waveform after detrend processing. The signal shaping processing section 123 generates a signal shaped into a waveform showing the period of stop and resumption of breathing by performing full-wave rectification processing, low pass filter application, and detrend processing. Also for the oxygen saturation signal, the signal shaping processing section 123 performs the detrend processing similarly. Thus, the cross-correlation analysis between the air flow signal after shaping and the oxygen saturation signal is possible. It is possible to calculate the LFCT by performing the cross-correlation analysis. Next, the cross-correlation analysis between the air flow signal after shaping and the oxygen saturation signal will be described with reference to FIG. 5.

FIG. 5 is a third diagram illustrating the outline of the circulation time measurement process in one embodiment of the present invention.

In FIG. 5, a graph 3C is a time-series graph of the air flow signal after shaping that is shaped into a waveform showing a period of stop and resumption of breathing. A graph 3D is a time-series graph of an oxygen saturation signal. A graph 3C-10 is a graph obtained by shifting the graph 3C by 10 seconds to the right. A graph 3C-20 is a graph obtained by shifting the graph 3C by 20 seconds to the right.

In order to measure the LFCT, the graph 3C or the graph 3D is shifted by a time difference corresponding to the time lag between both the graphs so that a predetermined time in the air flow signal after shaping and a time in the oxygen saturation signal, which indicates the behavior of the oxygen saturation corresponding to the resumption of breathing at the predetermined time, overlap each other. The shift amount (second) at this time is the LFCT. The LFCT is a time required for the delivery of oxygen to the fingertip of the subject. In order to calculate the shift amount at this time, the time difference calculation section 124 performs cross-correlation analysis. First, the time difference calculation section 124 shifts any one of the time-series graph of the air flow signal after shaping and the time-series graph of the oxygen saturation signal in the time axis direction, and calculates a product of values at each time of the two graphs after shift. The time difference calculation section 124 sums the product at each time for all times. The total value is referred to as a cross-correlation coefficient. The time difference calculation section 124 compares the cross-correlation coefficients calculated for each of the shift amounts, and calculates a shift amount when the value is the largest. The cross-correlation coefficient in this case is referred to as a maximum cross-correlation coefficient. That is, the time difference calculation section 124 calculates a shift amount in a case where the cross-correlation between the waveform of the graph 3C and the waveform of the graph 3D is the strongest. This processing is referred to as a cross-correlation analysis in the present embodiment. The shift amount calculated by the cross-correlation analysis is a time difference corresponding to the time lag between the waveform of the air flow signal after shaping and the waveform of the oxygen saturation signal, and is the LFCT.

In the case of the example shown in FIG. 5, the cross-correlation when the graph C is shifted by 0 seconds to the right (future direction) is low. When the graph C is shifted by 10 seconds to the right, the cross-correlation increases. When the graph C is shifted by 20 seconds to the right, the cross-correlation is the highest. In the case of the example shown in FIG. 5, the obtained LFCT is 20 seconds. That is, in this example, oxygen taken by breathing reaches a fingertip of the subject with a delay of 20 seconds. As described above, the delay time correlates with the indicator of the heart function of the subject. After the time difference calculation section 124 calculates the LFCT by the cross-correlation analysis, the cardiac output calculation unit 21 calculates an estimated value of a cardiac output CI using the LFCT. Next, the calculation of the cardiac output CI will be described.

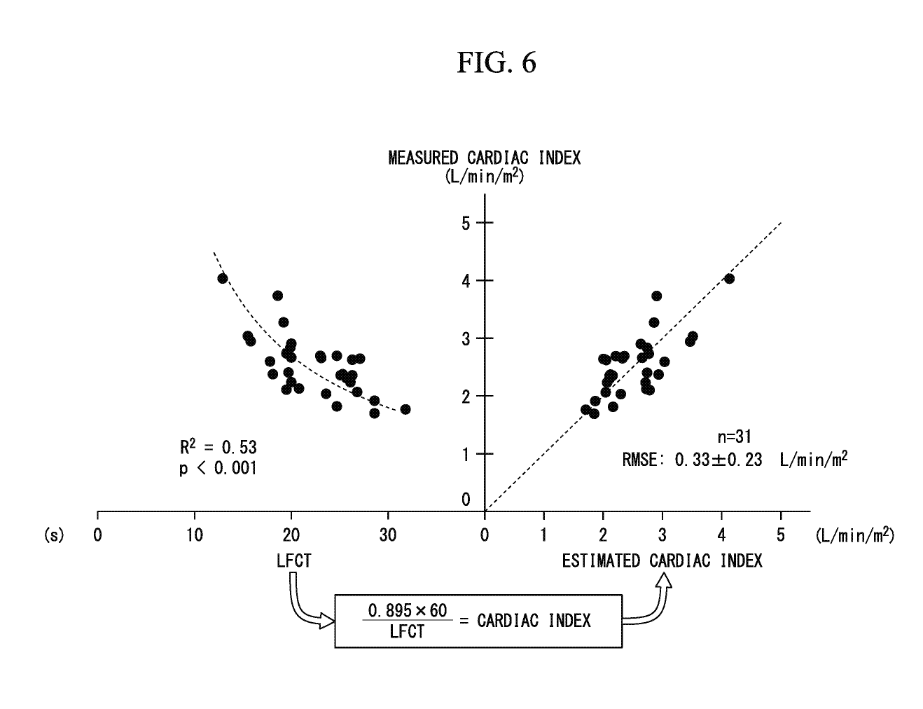

FIG. 6 is a diagram illustrating the calculation of a cardiac output in one embodiment of the present invention.

The left side of FIG. 6 is a graph showing the relationship between the LFCT according to the present embodiment measured for a plurality of subjects (31 persons) and the measurement value of the CI measured for the same subjects. In the left side of FIG. 6, the vertical axis indicates a CI measurement value, and the horizontal axis indicates the LFCT. The LFCT was measured using the air flow signal and the oxygen saturation signal measured overnight for a plurality of subjects, and the average value was adopted. For the measurement value of the CI, measurement was performed using the most accurate invasive measurement method (for example, a thermal dilution method or a Fick method) at present. R²=0.53 and p value<0.001 were obtained by performing regression analysis of the correlation between the average value of the LFCT and the CI measurement value obtained as described above. This can be said to be a meaningful value indicating there is a correlation between the average value of the LFCT and the CI measurement value. By analyzing the graph shown on the left side of FIG. 6, it can be seen that the relationship between the measurement values of the LFCT and the CI can be approximated to the relationship of the hyperbolic function. The relationship between the LFCT and the CI can be expressed by the following equation.

$$Cardiac index ( L \text{/} min \text{/} {m}^{2} ) = \frac{0.895 ( L \text{/} {m}^{2} ) \times 60 ( s \text{/} min )}{LFCT ( s )} \qquad ( 1 )$$

The right side of FIG. 6 is a graph showing the relationship between CI estimated values calculated for a plurality of subjects using the above Equation (1) and CI measurement values measured for the same subjects. On the right side of FIG. 6, the vertical axis indicates the CI measurement value, and the horizontal axis indicates the CI estimated value. By analyzing the graph shown on the right side, RMSE=0.33±0.23 (L/min/m²) was obtained for error RMSE (Root Mean Squared Error) of the CI estimated value according to Equation (1) of the present embodiment. This value is thought to be error that is allowable in use for medical purposes.

Based on the above analysis, in the present embodiment, the cardiac output calculation unit 21 acquires the LFCT measured by the circulation time measurement device 10, and calculates the estimated value of the cardiac output (CI) using Equation (1).

Next, the flow of the process of calculating the estimated value of the cardiac output (CI) in the present embodiment will be described.

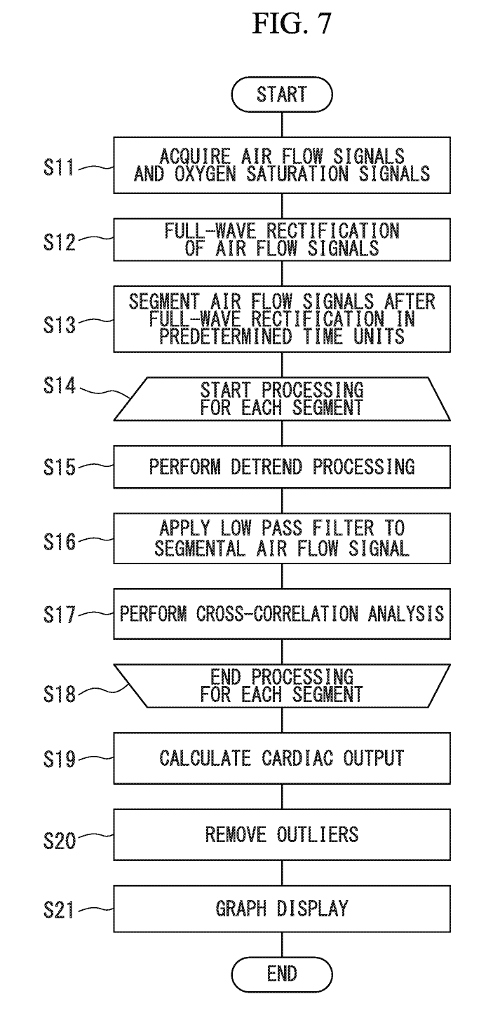

FIG. 7 is a flowchart of the processing of calculating the cardiac output according to one embodiment of the present invention.

As a prerequisite, a series of air flow signals and a series of oxygen saturation signals measured during sleep of the subject are stored in the storage unit 23. In addition, it is assumed that the measurement value of the pulse of the subject measured in parallel with the air flow signal or the like is stored in the storage unit 23.

First, the signal acquisition unit 11 reads and acquires a series of air flow signals and oxygen saturation signals of the subject from the storage unit 23 (step S11). The signal acquisition unit 11 outputs the read air flow signals and oxygen saturation signals to the circulation time calculation unit 12. Then, the signal shaping processing section 123 provided in the circulation time calculation unit 12 performs full-wave rectification processing on the read series of air flow signals (step S12). Then, the air flow segment section 121 provided in the circulation time calculation unit 12 generates N segmental air flow signals n (n=1 to N) by segmenting the air flow signals after full-wave rectification in predetermined time units (for example, 2-minute units) (step S13). In addition, the oxygen saturation segment section 122 provided in the circulation time calculation unit 12 generates N segmental oxygen saturation signals n (n=1 to N) by segmenting the series of oxygen saturation signals in the same time units (for example, 2-minute units) as the length used in the segment by the air flow segment section 121. The segmental air flow signal n and the segmental oxygen saturation signal n are referred to collectively as a segmental signal n. Then, the circulation time calculation unit 12 performs processing of the following steps S15 to S17 for each segment (step S14).

First, the signal shaping processing section 123 performs detrend processing on a first segmental air flow signal 1 and a first segmental oxygen saturation signal 1 (step S15). Then, the signal shaping processing section 123 removes high-frequency components by applying a low pass filter to the first segmental air flow signal 1 after detrend processing (step S16). At this time, the signal shaping processing section 123 applies a plurality of low pass filters to the first segmental air flow signal 1 after detrend processing. Examples of a plurality of types of low pass filters are shown.

A. First-order low pass filter, dead time=0, cutoff frequency 0.010 Hz

B. First-order low pass filter, dead time=0, cutoff frequency 0.015 Hz

C. First-order low pass filter, dead time=0, cutoff frequency 0.020 Hz

The signal shaping processing section 123 generates a shaped segmental air flow signal A1, a shaped segmental air flow signal B1, and a shaped segmental air flow signal C1 by applying the low pass filters A, B, and C to the first segmental air flow signal 1.

Then, the time difference calculation section 124 provided in the circulation time calculation unit 12 calculates a cross-correlation coefficient by performing cross-correlation analysis for the first segmental oxygen saturation signal 1 and each of the shaped segmental air flow signal A1, the shaped segmental air flow signal B1, and the shaped segmental air flow signal C1 (step S17). At this time, as an example of measuring the oxygen saturation signal at the fingertip of the subject, the time difference calculation section 124 performs cross-correlation analysis by limiting the shift amount of the shaped segmental air flow signal or the segmental oxygen saturation signal in the time axis direction to a range of, for example, 10 seconds to 60 seconds. The time difference calculation section 124 calculates a cross-correlation coefficient for each shift amount by shifting the shaped segmental air flow signal or the segmental oxygen saturation signal in a direction, in which points indicating the behaviors (a point indicating the resumption of breathing and a point indicating an increase in oxygen saturation) as marks of the shaped segmental air flow signal and the segmental oxygen saturation signal overlap each other, while changing the shift amount, and acquires a case where the value is the largest. Finally, the time difference calculation section 124 calculates a maximum cross-correlation coefficient A′1 between the shaped segmental air flow signal A1 and each segmental oxygen saturation signal. The time difference calculation section 124 calculates a maximum cross-correlation coefficient B′1 between the shaped segmental air flow signal B1 and each segmental oxygen saturation signal. The time difference calculation section 124 calculates a maximum cross-correlation coefficient C′1 between the shaped segmental air flow signal C1 and each segmental oxygen saturation signal. Then, the time difference calculation section 124 selects a maximum value D′1 among the calculated A′1, B′1, and C′1. Then, the time difference calculation section 124 sets the shift amount corresponding to the selected maximum value D′1 of the maximum cross-correlation coefficients as an LFCT1 for the segmental signal. The time difference calculation section 124 records a predetermined time included in the segmental air flow signal (for example, first measurement time of the air flow signal included in the segmental air flow signal) and the LFCT1 in the storage unit 23 so as to match each other.

The signal shaping processing section 123 and the time difference calculation section 124 repeat the processing of steps S15 to S17 similarly for the second segmental air flow signal and the second segmental oxygen saturation signal. For example, even if the time difference calculation section 124 selects the maximum cross-correlation coefficient C′1 for the first segmental signal and sets the shift amount in that case as the shift amount LFCT1, if the maximum cross-correlation coefficient A′2 is a maximum value in the cross-correlation analysis of the second segmental signal, the time difference calculation section 124 selects a shift amount in that case and sets the shift amount as a circulation time LFCT2 in the segmental signal. Thus, by applying a plurality of types of low pass filters to each segmental signal and comparing the results of processing by the low pass filters, it is possible to select the accurate LFCT (high maximum cross-correlation coefficient) for each segmental signal. After the LFCT2 is set, the time difference calculation section 124 records a predetermined time included in the second segmental air flow signal and the LFCT2 in the storage unit 23 so as to match each other.

By performing the processing on the segmental signal for all segmental signals (n=1 to N), time-series LFCTn (n=1 to N) is recorded in the storage unit 23. Then, the output unit 13 reads the time-series LFCTn (n=1 to N) from the storage unit 23, and outputs the LFCTn to the cardiac output calculation unit 21.

Then, the cardiac output calculation unit 21 calculates an estimated value CIn of the cardiac output for each of the time-series LFCTn (n=1 to N) using Equation (1) (step S19). The cardiac output calculation unit 21 records the calculated CIn in the storage unit 23 so as to match the time associated with the LFCTn. When the cardiac output calculation unit 21 completes the calculation of the CIn for all LFCTn (n=1 to N), time-series CIn (n=1 to N) is recorded in the storage unit 23.

Then, the cardiac output calculation unit 21 performs processing for removing outliers from the time-series CIn (step S20). For example, if there is a portion where the CIn has changed abruptly, the cardiac output calculation unit 21 removes the CIn that has changed abruptly. The case where the CIn changes abruptly is a case of ΔCI≧0.5 L/min/m², for example. In addition, if there is a value that significantly deviates from the average value of the CIn, the cardiac output calculation unit 21 removes the CIn. The significantly deviated value is a case where the amount of deviation is equal to or greater than 1.0 L/min/m², for example etc. The cardiac output calculation unit 21 deletes outliers from the data of the time-series CIn recorded in the storage unit 23.

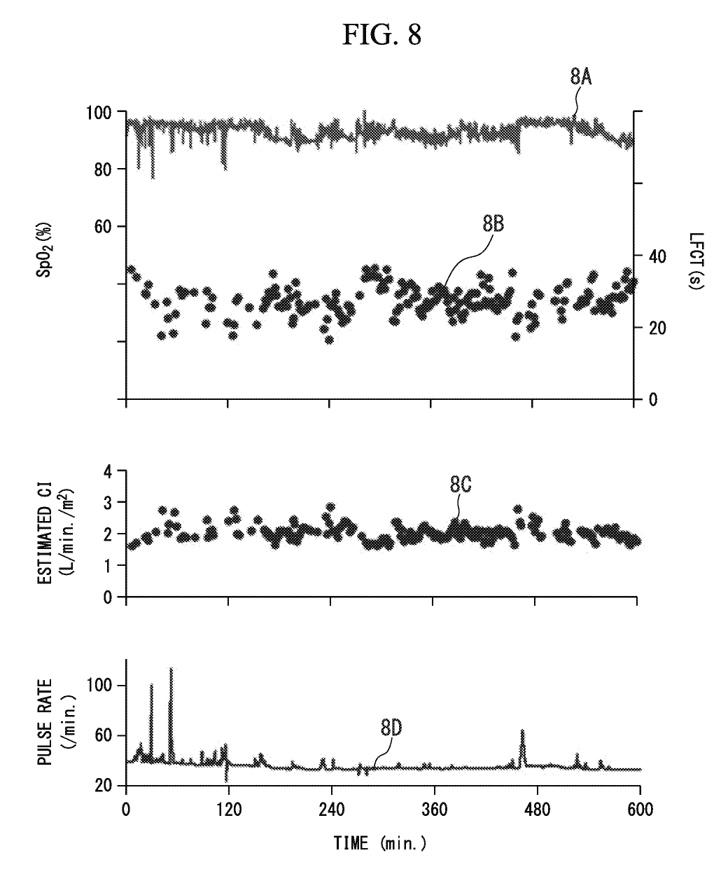

Then, the graph display unit 22 reads the CI estimated value and the time-series LFCT after removing the outliers, and generates an image in which time-series graphs of the LFCT, the CI estimated value, and the like are displayed. The graph display unit 22 outputs the generated image to the display device so as to be displayed on the display device (step S21). FIG. 8 shows an example of the graph output from the graph display unit 22.

FIG. 8 is a first diagram showing an example of the graph output from the estimated cardiac output calculation apparatus in one embodiment of the present invention.

A graph 8A (uppermost graph) shown in FIG. 8 is a time-series graph of oxygen saturation (SpO₂). A graph 8B (second graph from the top) is a time-series graph of the LFCT measured by the circulation time measurement device 10 of the present embodiment. A graph 8C (third graph from the top) is a time-series graph of the CI estimated value calculated by the estimated cardiac output calculation apparatus 20 of the present embodiment. A graph 8D (lowermost graph) is a time-series graph of the pulse. According to the present embodiment, the graphs exemplified in FIG. 8 can be output using the data measured by polysomnography.

The graph display unit 22 may calculate an average value from each of the time-series LFCT and the time-series cardiac output, and may output the average values.

In addition, although the case of calculating the estimated value of the CI as a cardiac output has been described as an example so far, the estimated value of the CO may be calculated. Specifically, information of the body surface area of the subject is recorded in the storage unit 23. Then, the cardiac output calculation unit 21 calculates a CI estimated value using Equation (1), and calculates a CO estimated value by multiplying the CI estimated value by the body surface area of the subject read from the storage unit 23 (CO estimated value=CI estimated value×body surface area of subject).

Although the processing for removing outliers is performed for the estimated value of CI in the process flow shown in FIG. 7, processing for removing a value that has changed abruptly or a value that significantly deviates from the average value may be performed for the measurement value of the time-series LFCT.

The full-wave rectification processing, the application of a low pass filter, the detrend processing, and the cross-correlation analysis processing that have been described above can be performed using general numerical analysis software, such as MATLAB provided by MathWorks, for example.

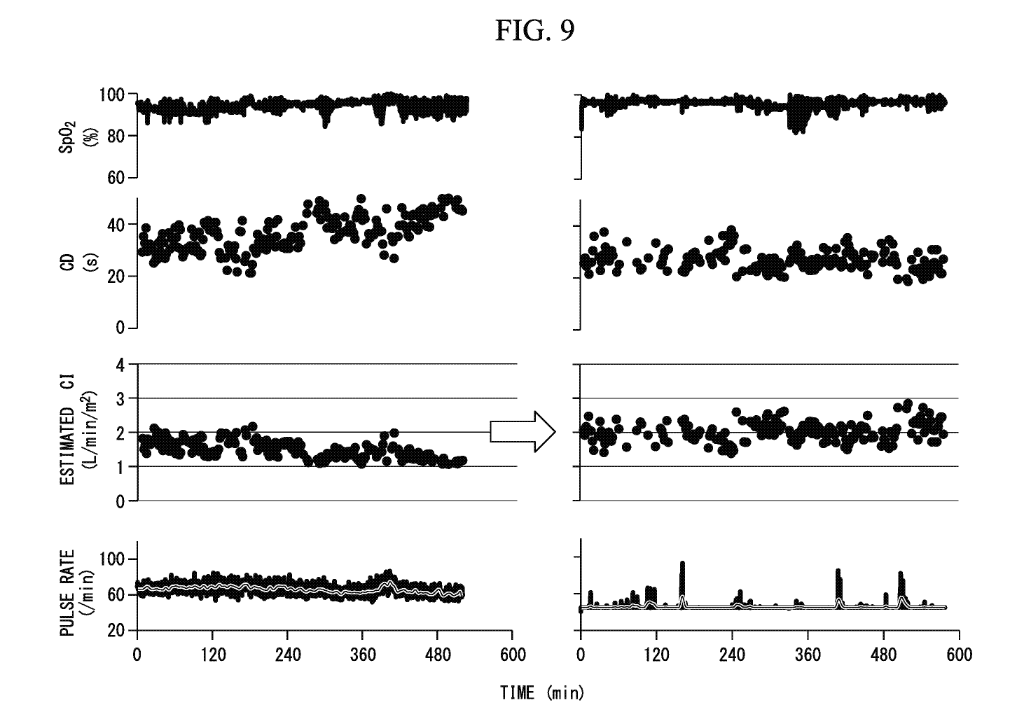

FIG. 9 is a second diagram showing an example of the graph output from the estimated cardiac output calculation apparatus in one embodiment of the present invention.

FIG. 9 is graphs showing the results of the measurement of the LFCT using the circulation time measurement device 10 of the present embodiment and the calculation of the CI estimated value using the estimated cardiac output calculation apparatus 20 for a certain subject suffering from atrial fibrillation and heart failure maintaining the contractile ability. The subject complained of dyspnea, and echocardiography was performed and a result indicating a good heart function was obtained. However, when the CI estimated value was calculated using the estimated cardiac output calculation apparatus 20 of the present embodiment, a reduction in the CI estimated value was observed (left side of FIG. 9).

Then, measures of electrical defibrillation were done for the subject, and then the CI estimated value was calculated again using the estimated cardiac output calculation apparatus 20 of the present embodiment when the symptoms were light. As a result, the right side of FIG. 9 was obtained. According to the right side of FIG. 9, it can be seen that the CI estimated value of the subject is recovering. This example shows that, even in a case where the state of the heart function of the subject cannot be detected by echocardiography or the like, it may be possible to grasp the state of the heart function using the estimated cardiac output calculation apparatus 20 of the present embodiment. In addition, since a patient with heart failure or the like also suffers from sleep-disordered breathing in many cases, the method of the present embodiment capable of estimating the CI, which is an important indicator of the heart function, based on the air flow signal and the oxygen saturation signal measured during sleep is also appropriate for a daily examination for a patient with heart failure or the like.

According to the estimated cardiac output calculation apparatus 20 of the present embodiment, it is possible to estimate the cardiac output CI in a non-invasive method based on the measurement value of the LFCT. In the measurement of the LFCT, neither a special device nor special skill is required. In addition, the measurement of the LFCT can be performed if an existing examination device and a PC or the like having a function of the circulation time measurement device 10 are present. Therefore, introduction and operation thereof are easy. In addition, in the field of day-to-day medical care, it is not practical to manually measure the LFCT from a large amount of data of the air flow signal and the oxygen saturation signal measured overnight for a plurality of subjects. According to the algorithm of the present embodiment, it is possible to automatically measure the LFCT by extracting the waveform showing the period of stop and resumption of breathing from the air flow signal and analyzing the cross-correlation with the oxygen saturation signal. Therefore, it is possible to continue the measurement of the LFCT on a daily basis without difficulty. In addition, according to the present embodiment, it is possible to display not only the LFCT or the CI estimated value at a single point in time but also graphs showing changes in the LFCT and the CI estimated value in a predetermined period (for example, overnight). Therefore, it is possible to obtain meaningful data regarding the subject.

In addition, the processing of each unit may be performed by recording a program for realizing all or some of the functions of the circulation time measurement device 10 and the estimated cardiac output calculation apparatus 20 in a computer-readable recording medium, reading the program recorded in the recording medium into a computer system, and executing the read program. The “computer system” referred to herein is intended to include an OS or hardware, such as a peripheral device.

In addition, the “computer system” may also include a homepage presenting environment (or a display environment) in a case where a WWW system is used.

In addition, examples of the “computer-readable recording medium” include portable media, such as a CD, a DVD, and a USB, and a storage device, such as a hard disk built into the computer system. The above program may be a program for realizing some of the functions described above or may be a program capable of realizing the above functions in combination with a program already recorded in the computer system.

It is also possible to appropriately replace the components in the above embodiment with known components without departing from the scope of the present invention. In addition, the technical range of the present invention is not limited to the embodiment described above, but various modifications can be made without departing from the spirit and scope of the present invention. For example, the storage unit 23 may be provided in an external storage device. The LFCT is an example of the oxygen delivery circulation time of blood. In addition, Equation (1) is an example of a predetermined hyperbolic function showing the relationship between the oxygen delivery circulation time of blood and the cardiac output. The estimated value of the CI is an example of the estimated cardiac output.

### INDUSTRIAL APPLICABILITY

According to the circulation time measurement device, the estimated cardiac output calculation apparatus, the circulation time measurement method, the estimated cardiac output calculation method, and the program described above, it is possible to measure the oxygen delivery circulation time of blood and estimate the cardiac output using the breathing period of the subject and the time-series data of oxygen saturation in blood.

### REFERENCE SIGNS LIST

10: circulation time measurement device
11: signal acquisition unit
12: circulation time calculation unit
121: air flow segment section
122: oxygen saturation segment section
123: signal shaping processing section
124: time difference calculation section
13: output unit
20: estimated cardiac output calculation apparatus
21: cardiac output calculation unit
22: graph display unit
23: storage unit

1-9. (canceled)

10. A circulation time measurement device, comprising:
a signal acquisition unit that acquires an air flow signal indicating a temporal change of an air flow of breathing and an oxygen saturation signal indicating a temporal change of oxygen saturation; and
a circulation time calculation unit that measures an oxygen delivery circulation time of blood based on a time difference between a predetermined first time in the air flow signal and a second time in the oxygen saturation signal indicating an increase in oxygen saturation corresponding to resumption of breathing at the first time,
wherein the circulation time calculation unit includes:
an air flow segment section that segments the air flow signal at predetermined time intervals to generate a segmental air flow signal;
an oxygen saturation segment section that segments the oxygen saturation signal at the predetermined time intervals to generate a segmental oxygen saturation signal;
a signal shaping processing section that generates a shaped segmental air flow signal by shaping the segmental air flow signal into a waveform showing a period of stop and resumption of breathing by applying a filter to the segmental air flow signal; and
a time difference calculation section that calculates a time difference corresponding to a time lag between a waveform indicated by the shaped segmental air flow signal and a waveform indicated by the segmental oxygen saturation signal and sets the time difference as the oxygen delivery circulation time.

11. The circulation time measurement device according to claim 10,
wherein the time difference calculation section calculates the time difference using a cross-correlation analysis for the shaped segmental air flow signal and the segmental oxygen saturation signal.

12. An estimated cardiac output calculation apparatus, comprising:
the circulation time measurement device according to claim 10, and
a cardiac output calculation unit that acquires the oxygen delivery circulation time measured by the circulation time measurement device and calculates an estimated cardiac output based on the acquired oxygen delivery circulation time and a predetermined hyperbolic function showing a relationship between the oxygen delivery circulation time of blood and a cardiac output.

13. A circulation time measurement method, comprising:
a step of acquiring an air flow signal indicating a temporal change of an air flow of breathing and an oxygen saturation signal indicating a temporal change of oxygen saturation; and
a step of measuring an oxygen delivery circulation time of blood based on a time difference between a predetermined first time in the air flow signal and a second time in the oxygen saturation signal indicating an increase in oxygen saturation corresponding to resumption of breathing at the first time,
wherein, in the step of measuring the oxygen delivery circulation time, a segmental air flow signal is generated by segmenting the air flow signal at predetermined time intervals;
a segmental oxygen saturation signal is generated by segmenting the oxygen saturation signal at the predetermined time intervals;
a shaped segmental air flow signal is generated by shaping the segmental air flow signal into a waveform showing a period of stop and resumption of breathing by applying a filter to the segmental air flow signal; and
a time difference corresponding to a time lag between a waveform indicated by the shaped segmental air flow signal and a waveform indicated by the segmental oxygen saturation signal is calculated, and the time difference is set as the oxygen delivery circulation time.

14. An estimated cardiac output calculation method, comprising:
acquiring an air flow signal indicating a temporal change of an air flow of breathing and an oxygen saturation signal indicating a temporal change of oxygen saturation;
measuring an oxygen delivery circulation time of blood based on a time difference between a predetermined first time in the air flow signal and a second time in the oxygen saturation signal indicating an increase in oxygen saturation corresponding to resumption of breathing at the first time; and
calculating an estimated cardiac output based on the measured oxygen delivery circulation time and a predetermined hyperbolic function showing a relationship between the oxygen delivery circulation time of blood and a cardiac output.

15. A program causing a computer of a circulation time measurement device to function as:
a signal acquisition unit that acquires an air flow signal indicating a temporal change of an air flow of breathing and an oxygen saturation signal indicating a temporal change of oxygen saturation, and
in measuring an oxygen delivery circulation time of blood based on a time difference between a predetermined first time in the air flow signal and a second time in the oxygen saturation signal indicating an increase in oxygen saturation corresponding to resumption of breathing at the first time, to function as:
an air flow segment section that segments the air flow signal at predetermined time intervals to generate a segmental air flow signal;
an oxygen saturation segment section that segments the oxygen saturation signal at the predetermined time intervals to generate a segmental oxygen saturation signal;
a signal shaping processing section that generates a shaped segmental air flow signal by shaping the segmental air flow signal into a waveform showing a period of stop and resumption of breathing by applying a filter to the segmental air flow signal; and
a time difference calculation section that calculates a time difference corresponding to a time lag between a waveform indicated by the shaped segmental air flow signal and a waveform indicated by the segmental oxygen saturation signal and sets the time difference as the oxygen delivery circulation time.

16. A program causing a computer of an estimated cardiac output calculation apparatus to function as:
a signal acquisition unit that acquires an air flow signal indicating a temporal change of an air flow of breathing and an oxygen saturation signal indicating a temporal change of oxygen saturation;
a circulation time calculation unit that measures an oxygen delivery circulation time of blood based on a time difference between a predetermined first time in the air flow signal and a second time in the oxygen saturation signal indicating an increase in oxygen saturation corresponding to resumption of breathing at the first time; and
a cardiac output calculation unit that calculates an estimated cardiac output based on the measured oxygen delivery circulation time and a predetermined hyperbolic function showing a relationship between the oxygen delivery circulation time of blood and a cardiac output.

17. An estimated cardiac output calculation apparatus, comprising:
the circulation time measurement device according to claim 11; and
a cardiac output calculation unit that acquires the oxygen delivery circulation time measured by the circulation time measurement device and calculates an estimated cardiac output based on the acquired oxygen delivery circulation time and a predetermined hyperbolic function showing a relationship between the oxygen delivery circulation time of blood and a cardiac output.
